(19) United States
(12) Patent Application Publication
(10) Pub. No.: US 2005/0117693 A1
(43) Pub. Date: Jun. 2, 2005

(54) Tomograph

(76) Inventor: Iwao Miyano, Nagareyama-shi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/509,915

(22) PCT Filed: Apr. 3, 2003
(86) PCT No.: PCT/JP03/04286

(30) Foreign Application Priority Data
Apr. 4, 2002 (JP) 2002103124

(51) Int. Cl. G21K 1/12; H05G 1/60; G01N 23/00; A61B 6/00

(52) U.S. Cl. 378/4

(57) ABSTRACT

An X-ray CT apparatus takes a short time for image processings in irradiating a cone beam X-ray to the whole jaw, including a dental arc, and to a local region, and to obtain a panoramic image showing conditions of a dental row. The apparatus includes, a rotative arm suspended at one end of a support column, and an X-ray generating device and a two-dimensional X-ray detecting device oppositely fixed to respective ends of the rotative arm. The rotative arm is supported via a dual rotation system. An object to be examined sits on a chair which is movable upward and downward, and an examining region is adjusted to the height of the imaging center of the imaging apparatus. The object's head is fixed by a fine-adjustable head holder. Imaging is performed so that the distance between rotation centers of the two rotation systems substantially coincides with the object's dental arc.

### TECHNICAL FIELD

The present invention relates to an X-ray CT apparatus for obtaining a tomographic image and the like by irradiating an X-ray to a portion of an object to be examined and processing a projection image of the region; and, more particularly, the invention relates to an X-ray CT apparatus which can obtain an arbitrary CT image and a panoramic image of a region by irradiating a cone beam X-ray, which is suitable for imaging in dental examination.

### BACKGROUND OF THE INVENTION

In current dental examination, general imaging in which a film is held at the back of teeth to perform X-ray imaging, panoramic imaging in which an X-ray tube and a film are simultaneously revolved, cephalometric imaging in which an X-ray tube is greatly detached from a film during imaging, and the like are performed. The X-ray panoramic imaging for dentistry is an imaging method in which curved cross sections are sequentially imaged along a tooth row, and the tomographic images are spread out and displayed as one panoramic image showing conditions of the tooth row and tissue and bone around it.

In a conventional panoramic imaging apparatus, a rotative arm mounting an X-ray generating device and a two-dimensional X-ray detecting device, which are disposed so as to face each other, while interposing the object therebetween, is supported by, e.g. a back-forth/left-right movable unit and a rotative unit, which is designed to move around the object along a complicated orbit simulating the shape of a dental arc therebetween. An example of a tomograph for dentistry of this kind is disclosed in Japanese Unexamined Patent Publication No.Hei06-78919.

Also, as a dental X-ray imaging apparatus, an X-ray CT apparatus which can obtain a horizontal tomographic image of a single tooth, other than a panoramic image, has been proposed. The apparatuses of this kind include, for example, a medical X-ray tomograph of the type mentioned in Japanese Unexamined Patent Publication No.Hei 09-122118 (the first conventional technique) and a panoramic X-ray imaging apparatus of the type mentioned in Japanese Unexamined Patent Publication No.Hei 11-318886 (the second conventional technique). A known example of a general medical CT apparatus using a cone beam X-ray is mentioned in Japanese Unexamined Patent Publication No.Hei 10-192267 (the third conventional technique). According to this apparatus, a tomographic image covering a wide region of the object can be obtained so as to be applicable to imaging on the jaw, including the dental arc.

Meanwhile, a technique involved with an X-ray CT imaging method and apparatus for irradiating a cone beam X-ray only to one portion of the object and obtaining an arbitrary tomographic image or a three-dimensional image of the portion is mentioned as an example of a local irradiation X-ray CT apparatus in Japanese Unexamined Patent Publication No.2000-139902 (the fourth conventional technique). Specially, in a dental application, a cone beam X-ray is irradiated with rotation not to the whole jaw including the dental arc, but only to a local region limited around a tooth and jaw joint, to thereby reduce the exposure dose, and a CT image and a three-dimensional image of high resolution are provided.

It is known that in any of the above-described conventional techniques, it takes a very long time (20 minutes to about one hour) for image calculation processing after imaging until an image is presented on an image display device.

The present invention has been developed in consideration of the above described points, and its object is to provide an X-ray CT apparatus which can greatly shorten the time taken for image processing when a panoramic image showing conditions of a tooth row, teethridge, and tissue and bone around them is obtained.

### SUMMARY OF THE INVENTION

To achieve the above-stated object, an X-ray CT apparatus according to the present invention includes X-ray generating means for generating an X-ray, X-ray detecting means for two-dimensionally detecting an X-ray dose which is transmitted through an object to be examined, holding means for holding the X-ray generating means and the X-ray detecting means so that the object is located therebetween, first rotation driving means for driving the holding means to rotate the X-ray generating means and the X-ray detecting means around the object, containing means attached to the holding means for containing the first rotation driving means, image processing means for producing an image involved with the object on the basis of the X-ray dose detected by the X-ray detecting means, and image display means for displaying an image created by the image processing means. The X-ray CT apparatus further includes second rotation driving means for rotating, as one body, the holding means and the containing means, wherein the second rotation driving means contained in the containing means is in parallel with a rotation center of the first rotation driving means and in a different relation of the rotation center position than that of the first rotation driving means, and drive control means for controlling the driving of the first rotation driving means in a first imaging mode and separately driving the second rotation driving means and the second driving means in a second imaging mode.

The first rotation driving means is designed to rotate the holding means for holding the X-ray generating means and the X-ray detecting means, which are arranged opposite to each other, with respect to the object. An X-ray is irradiated while the X-ray generating means and the X-ray detecting means rotate around a local region in the vicinity of the rotation center of the first rotation driving means. By the second rotation driving means, the holding means and the first rotation driving means are driven and rotate as one body, whereby the rotation center of the first rotation driving means is revolved on a predetermined circle. Then in the first imaging mode (CT imaging), the location of a local X-ray irradiating region is determined by moving the rotation center of the first rotation driving means on the approximate circumference of a dental arc using the second rotation driving means. In the second imaging mode (panoramic imaging), the rotation center of the first rotation driving means moves along the approximate circumference of the shape of the dental arc, while the rotation angle, i.e. the imaging direction can be properly adjusted so that the irradiating direction is substantially perpendicular to the dental arc.

Because a most suitable panoramic image can be obtained in this way by using a simple mechanical means, complicated image calculating processing becomes unnecessary, whereby the time necessary for image processing in obtaining a panoramic image can be greatly shortened. When the distance between the X-ray generation source and the object varies due to the difference between the shape of the locus of movement and that of the actual dental arc, so that the expansion ratio of the fluoroscopic image varies depending on the tooth position, a proper panoramic image can be obtained by correcting the variation in the image calculating processing on each of the acquired local data in synchronism with the rotation angle of the rotation mechanism moving along the approximate circumference of the dental arc and reconstructing the whole image.

### BRIEF DESCRIPTION OF THE DRAWINGS

FIG. 1 is a diagrammatic side view showing the structure of an X-ray CT apparatus according to the present invention, and also showing a cross sectional structure of one portion.

FIG. 2 is a partial enlarged view of the cross sectional structure of the one portion shown in FIG. 1 for easy understanding.

FIG. 3 is a diagram showing a procedure used for positioning in a case where imaging is performed by the X-ray CT apparatus according to the embodiment of FIG. 1.

FIG. 4 is a partial enlarged view of FIG. 3.

FIG. 5 is a diagram showing an operation in a case where panoramic imaging is performed by the X-ray CT apparatus according to the embodiment of FIG. 1.

FIG. 6 is a partial enlarged view of FIG. 5.

FIG. 7 is a diagram showing differences between the rotation center of a first rotation system 6 and centers of teeth in a case where the rotation angle of the second rotation system is changed at every irradiation.

FIGS. 8a and 8b are diagrams respectively showing the state before and after correction of an expansion ratio of fluoroscopic images taken by each cone beam X-ray in the arrangement of FIG. 7.

FIG. 9 is a top view of a variation of a rotation mechanism of the second rotation system in the X-ray CT apparatus according the present invention.

FIG. 10 is a diagram showing a rotation radius drawn by the X-ray CT apparatus of FIG. 9.

FIG. 11 is a diagrammatical sectional view of the structure of a positioning device used in the X-ray CT apparatus according to this embodiment.

### BEST MODE FOR CARRYING OUT THE INVENTION

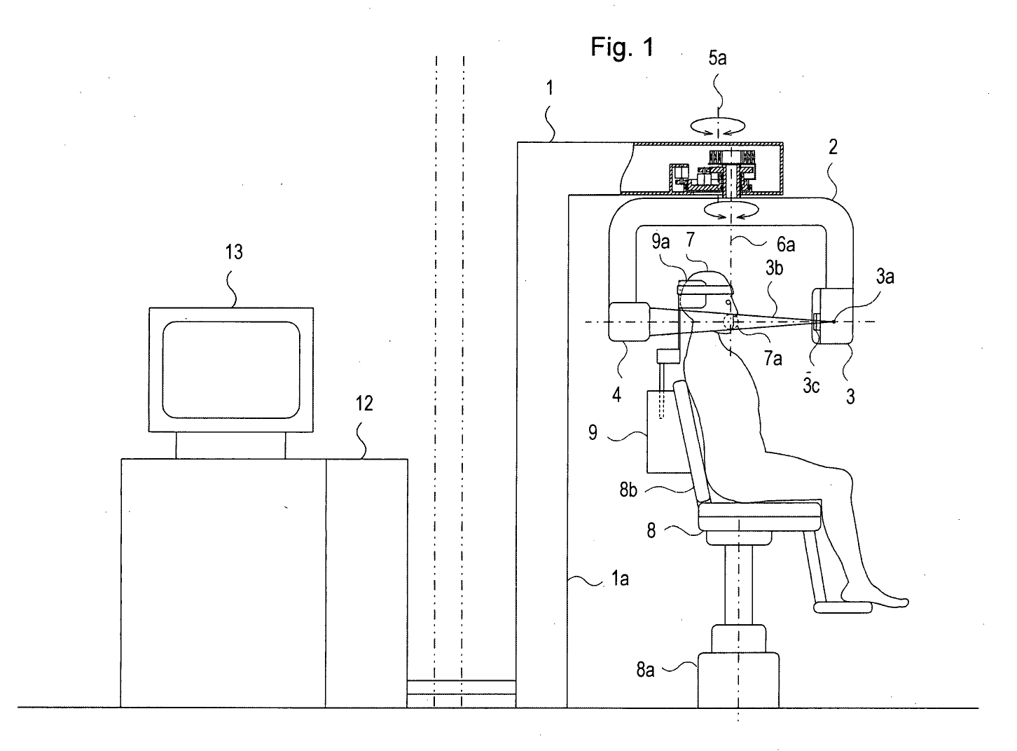

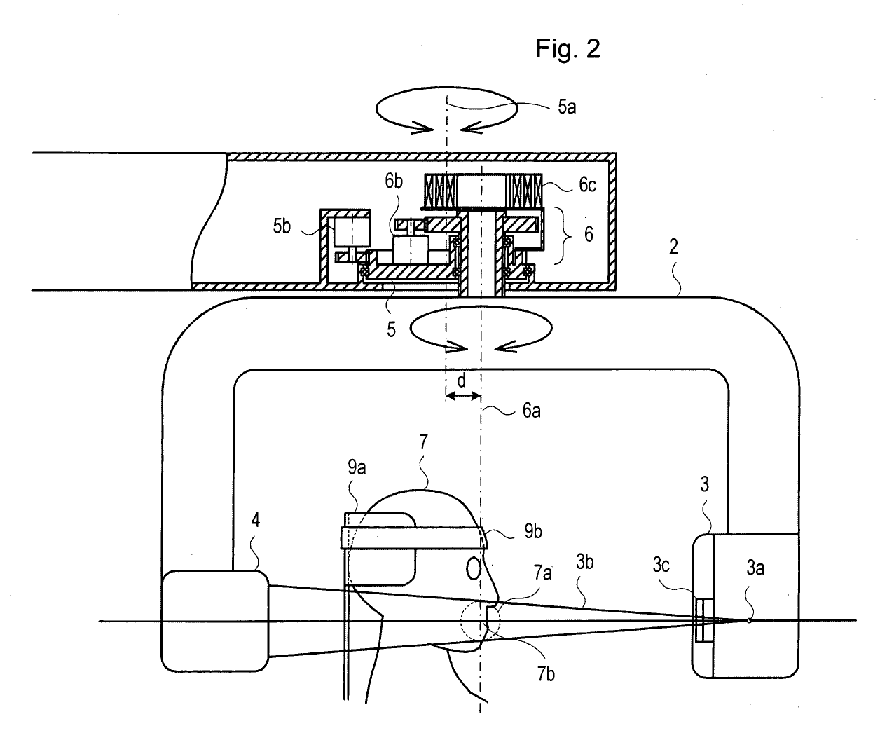

Hereinafter, a preferable embodiment of an X-ray CT apparatus according to the present invention will be described with reference to the accompanying drawings. FIG. 1 is a side view showing an example of the structure of the X-ray CT apparatus according to the present invention, which also shows the structure of a cross section of a region in which the rotation system is disposed. FIG. 2 is a partial enlarged view showing the partial cross sectional structure of FIG. 1 for easy understanding.

This X-ray CT apparatus includes a fixing column 1, a rotative arm 2, an X-ray generating device 3, a two-dimensional X-ray detecting device 4, a first rotation system 6, a second rotation system 5, a chair 8, and a head holder 9. Fixing column 1, being in a reverse L-shape, is supported by column portion 1a and houses the second rotation system 5 and first rotation system 6 at one end thereof. Rotative arm 2 is suspended from the end of the fixing column 1. First rotation system 6 holds the rotative arm 2 in the suspended state and rotates the arm 2 at a predetermined speed around the rotation center of the rotation shaft 6a at the end of fixing column 1. The second rotation system 5 is designed to rotate the whole first rotation system 6 at a predetermined speed around rotation axis 5a. That is, the positional relationship between the second rotation system 5 and the first rotation system 6 is such that they are arranged in parallel and each have a different rotational center, although the rotation systems are contained in a common containing unit. The second rotation system 5 and the first rotation system 6 will be described later in more detail.

Although the second rotation system 5, the first rotation system 6, and the rotative arm 2 attached thereto are arranged over the head of object 7 in the above-described example, they also may be arranged in the direction of the feet of object 7. By doing so, when object 7 sits on the chair in accordance with the imaging position of the X-ray CT apparatus, the object 7 need not worry about collision with the rotation system, including the rotative arm 2.

The X-ray generating device 3, which is designed to generate an X-ray, is located on one end of rotative arm 2. The X-ray generating device 3 includes a collimator device 3c for narrowing down the X-rays 3b irradiated from the X-ray generation source 3a inside the X-ray generating device 3 into a cone-shaped beam. The two-dimensional X-ray detecting device 4, which is arranged opposite to X-ray generating device 3, is designed to two-dimensionally detect an X-ray dose transmitted through the object, and it is installed in the other end of the rotative arm 2. That is, the X-ray generating device 3 and the two-dimensional X-ray detecting device 4 are arranged opposite to each other on respective ends of the rotative arm 2. Rotative arm 2 is driven by the first rotation system 6 to rotate it by approximately 405° around a rotation center near the tip of the fixing column 1. Although the imaging range is 360° , the rotation range is wider than it this 45°, because imaging is started when the rotation speed becomes constant. The rotation range is widely set for accelerating the rotation until the rotation of the first rotation system 6 becomes constant and decelerating the rotation until the first rotation system 6 stops after imaging, and so it is not limited to 45°. After imaging is started, the X-ray generating device 3 irradiates an X-ray in pulse form in synchronism with image acquisition, and X-ray exposure to the object is thus reduced. The timing thereof is controlled by a positional detection encoder built into the first rotation system 6. Inside column portion 1a of the fixing column 1, a control system for control of the imaging apparatus is installed.

Image data acquired by the two-dimensional X-ray detecting device 4 is sent to an image processing device 12. The image processing device 12 is installed in an operation room distant from the imaging room in which the X-ray CT apparatus is installed. Image processing device 12 performs calculation processing on the received image data, reconstructs a two-dimensional tomographic image, a CT image, or a three-dimensional image, and presents the image on the image display device 13. Image display device 13 includes an input device, such as a keyboard and a mouse (not shown), and it operates so that the image processing device 12 functions as a computer device. Because conditions of image reconstruction can be input from this input device, it is possible to input which of the two-dimensional tomographic image, CT image, the three-dimensional image, and the panoramic image is to be a subject of the reconstruction.

Rotative arm 2 is supported rotatably and horizontally relative to the fixing column 1. In this embodiment, the rotative arm 2 is constructed to be an up-down dual structure, including second rotation system 5 and first rotation system 6. Second and first rotation systems include a rotation supporting mechanism using a bearing, a driving mechanism and a position detecting mechanism rotated by the combination of a servomotor and a gear, and a cable processing mechanism of the rotation unit. The rotation center 5a of the second rotation system 5 is fixed relative to the fixing column 1 and the rotation center 6a of the first rotation system 6 is fixed relative to the rotative arm 2. The rotation centers 5a and 6a are spaced from each other by a fixed distance d. Rotation center 6a of the first rotation system 6 is rotated by a driving device 5b of the second rotation system 5 around a rotation axis on the rotation center 5a of the second rotation system 5. And, driving device 5b (including the position detecting device) of the second rotation system 5 and a cable processing mechanism (concrete structure being not shown) are contained inside the fixing column 1. The driving device 6b of the first rotation system (including the position detecting device) and the cable processing mechanism 6c are contained in the upper part of the second rotation system 5. The distance d between the two rotation centers 5a and 6a substantially corresponds to the size of the dental arc of object 7, e.g. a diameter of around 70 to 100 mm. The first rotation system 6 has to be rotated by 360° or more (around 405°) to acquire CT image data. On the other hand, because the second rotation system 5 is provided for the purpose of making a rotation similar to the dental arc, it is sufficient for it to rotate ±120° at the maximum.

Further, because there are individual differences in remaining teeth and the like of the object 7, the maximum rotation angle of the second rotation system 5 is not limited to ±120°, and it can be arbitrarily set using an input device.

The cable processing mechanism of the second rotation system 5 and the first rotation system 6 is commonly provided by using a guiding rail along the movement of the cable caused by the rotations. Accordingly, a plurality of cable processing mechanisms are unnecessary, and so the installation space of the mechanism can be miniaturized.

Meanwhile, object 7 sits on the chair 8, which can be moved upward and downward. The position of examining region 7a of the object 7 is determined relative to the height of the imaging center of the imaging apparatus. The angle of the chair back 8b of the chair 8 can be adjusted at an arbitrary angle. The position of object 7 in a back-front direction is substantially adjusted by combining the angle adjustment of the chair back 8b and the position adjustment effected by up-down movement provided by the mechanism 8a. A head holder 9 is provided in the rear of the chair back 8b and is adjustable upward and downward, backward and forward, and left and right in accordance with the seated height of the object 7 and the position of the examining region, so as to fix the head of the object 7 to a desirable position after adjustment of the chair 8. The head of the object 7 is moved to a desirable position by an operator and fixed by head band 9b or the like. The center of the examining region 7a of the object 7 can be adjusted to the rotation center 6a of the rotative arm 2 (rotation center of first rotation system 6 mentioned above) by the operator.

Chair 8 is moved upward and downward by the up-down movement mechanism 8a. If the head holder 9 is attached to the angle-adjustable chair back 8b, it is not necessary to use a dedicated chair according to the embodiment. That is, if the specifications of the up-down stroke and the like are fulfilled, a chair of the type used, e.g. by a hairdresser, may be utilized. Further, a therapeutic chair of the type used in otorhinolaryngology also may be utilized.

Further, as seen in FIG. 1, a state in which the back of object 7 is directed to column 1a, which is perpendicular to the floor on which fixing column 1 is installed, is shown. However, because the imaging apparatus is separated from the chair in this embodiment, the angle of the object's position 20 relative to column 1a is not limited as long as a rotation range resides in CT imaging. For example, imaging also can be performed while the object 7 is placed so as to face the column 1a. In this case, by setting a projector of an optical marker for positioning on the column, the optical marker can be projected from a direction opposite to object 7, whereby positioning becomes easy. Alternatively, the setting direction of column 1a and chair 8 can be freely set in relation to the layout of the imaging room where the apparatus is installed.

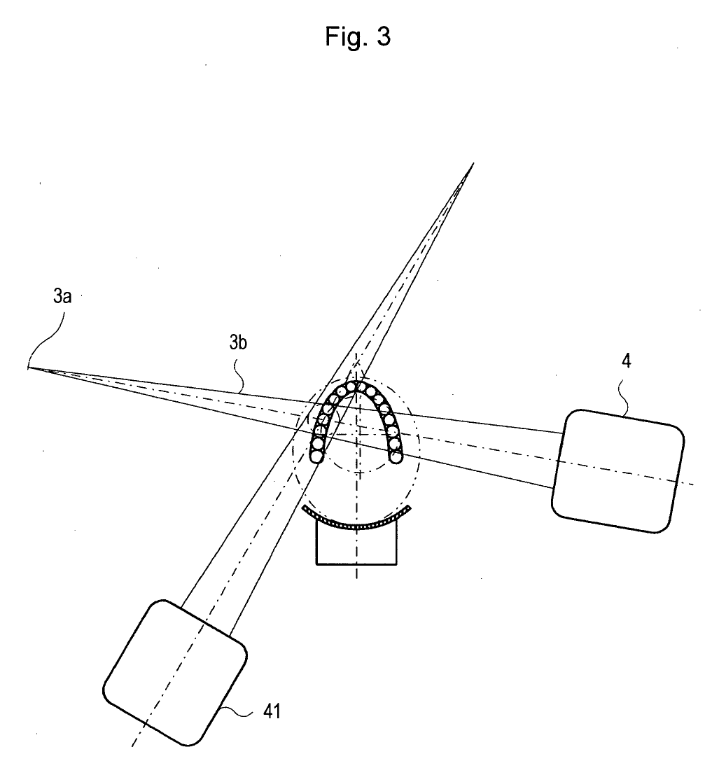

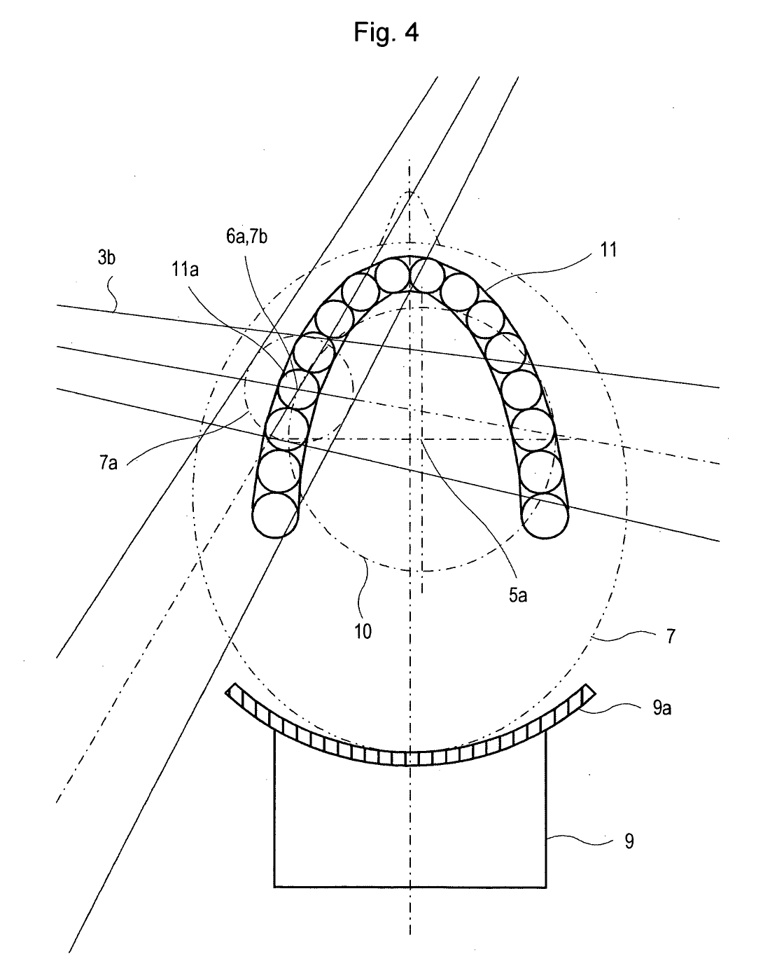

FIG. 3 is a diagram showing the procedure for positioning the object when imaging is performed by the X-ray CT apparatus according to this embodiment, and FIG. 4 is a partial enlarged view of FIG. 3. When CT imaging is performed locally on one or two teeth only, the second rotation system 5 is revolved so as to match center 7b of the region where the tooth 11 a to be imaged is located, using the rotation center 6a of first rotation system 6 (rotative arm) of the X-ray CT apparatus. However, circle 10, the rotation radius of which corresponds to the constant distance d between the rotation center 5a of the second rotation system 5 and the rotation center 6a of the first rotation system 6, is made substantially to coincide with the shape and size of dental arc 11. Accordingly, circle 10 does not always coincide with dental arc 11 depending on the position of the tooth being examined. That is, the center 7b of the examining region cannot be matched with the center 6a of the rotative arm only by making object 7 sit on the center in the left-right direction of the chair 8.

If imaging region 7a has a sufficient size to include several teeth, so that the examining region is located in the vicinity of the center of the imaging range 7a, imaging is sufficiently performed even when the center 7b of the examining region is spaced a little from the center 6a of the rotative arm. However, when the imaging region is limited to one or two teeth because of a limitation of the detectable size of the two-dimensional X-ray detecting device 4, or when the shape and size of the dental arc 11 is individually different as between an adult and a child, so that apparent differences occur between the shape and size of the circle 10, whose rotation radius corresponds to the distance d between rotation centers 5a and 6b of the second and rotation system 5 and the first rotation system 6 in the X-ray CT apparatus according to this embodiment, and those of the object's dental arc, resulting in the tooth 11a possibly being out of the imaging region 7a, it is necessary to match the center 7b of the examining region and the rotation center 6a of the rotative arm as accurately as possible.

According to this embodiment, the object's position in the back-front direction is adjusted by properly combining the angle of adjustment of the chair back 8b of the chair 8 and the up-down position adjustment by the up-down movement mechanism 8a to substantially match center 7b of the examining region 11a with the rotation center 6a of the first rotation system 6 (rotative arm 2). After fixing the head of the object to the head holder 9a of the chair 8, the head position is fine adjusted by using the back-front and left-right movement mechanism of the head holder 9. In this manner, the center 7b of the examining region 11a can be completely matched with the center 6a of the first rotation system 6 (rotative arm 2).

In the above-described positioning procedure, the positioning is generally performed with reference to a linear optical marker projected to the body surface of the object 7 in the state in which the object's mouth cavity is closed. Therefore, in some cases, it is difficult to check from outside whether or not the position of the tooth imaged completely coincides with the center of the imaging range.

In this case, a more accurate method of positioning is applicable, in which positioning is performed using an optical marker from outside, as described above, the direction of the rotative arm 2 of the imaging apparatus is then changed, and X-ray fluoroscopic imaging is performed from two orthogonal directions, whereby the position of the object 7 is remotely and finely adjusted, while the position of the teeth is visually checked on a fluoroscopic image. In this case, the position adjustment can be carried out more precisely and accurately by directly finely adjusting the position of the head of object 7 by remotely controlling the head holder 9, rather than by performing fine adjustment while moving the object 7. Further, from the viewpoint of safety, too, the distance of movement of the object 7 is desirably kept to a minimum in order to prevent the object 7 from touching the X-ray CT apparatus, and to reduce any external force applied to the object 7 while moving the object 7. In the case of the X-ray CT apparatus according to this embodiment, it is characteristic that, after adjusting the rough position of the object in the apparatus, the head holder of the chair 8 is separately finely adjusted. According to this embodiment, the range of fine adjustment to the head is around ±15 mm at most. Accordingly, the burden on the object caused by movement of the head holder 9 is much smaller in comparison with the case in which the position adjustment is carried out by moving the entire object (±50 mm to the maximum). Furthermore, it is possible to improve the accuracy of positioning and to shorten the time taken therefor.

Further, other than positioning using the rotation mechanism, it is also applicable to use the second rotation system only in panoramic imaging, and positioning of the object 7 is performed by combining up-down movement of the chair and up-down, back-forth, and left-right movement of the head holder 9.

Since the rotative arm 2 rotates around the head holder 9 to irradiate a cone beam X-ray, head holder 9a to which the cone beam X-ray 3b is irradiated is desirably made of a material which is permeable to radioactive rays and has enough strength to hold and fix the head, such as carbon fiber, so that the head holder 9a does not become an obstacle to image data acquisition by reason of the fact that X-rays are absorbed thereby.

An advantage of using the head holder 9 is that, since the back of the head of the object 7 is fixed by the head holder 9a, safety in a region where the object 7 cannot visually check (i.e. at the back of the head) by himself/herself is ensured, if it is ensured in the apparatus that the head holder 9a does not touch the main body of the X-ray CT apparatus during rotation of the rotative arm 2.

Although only the head holder 9 is used as means for fixing and positioning the object 7 in this embodiment, the fixing and positioning means is not limited to adjustment of the head holder 9. It is possible to utilize a combination of a chin rest and an ear rod, or a fixing device using a dental articulation model produced in accordance with a denture model of each object, in combination with the head holder. If those devices are constructed so that it can be fine adjusted back and forth, and left and right, a similar positioning function can be realized by fine adjusting their position.

In the state in which the position of the object 7 is fixed, as described above, CT imaging is performed by revolving the rotative arm 2, while cone beam X-ray 3b is irradiated from the X-ray generating device 3. In accordance with the rotation angle of rotative arm 2, the two-dimensional X-ray detecting device 4 mounted opposite to the X-ray generating device 3 (not shown) at the other end of rotative arm 2 is rotated from the position of two-dimensional X-ray detecting device 4 to that of two-dimensional X-ray detecting device 41. Eventually, fluoroscopic image data over 360° of diagnostic region 11a is acquired. The acquired image data is subjected to calculation processing at image processing device 12, a two-dimensional tomographic image or a three-dimensional image is reconstructed, and the image is displayed on the image display device 13.
The execution of the above-described imaging process can be outlined in the following order of steps (1) to (5):(1) An imaging region of object 7 is substantially positioned by rotating the second rotation system 5.
(2) The position of object 7 is fixed by finely adjusting the head holder 9 of chair 8.
(3) If necessary, X-ray fluoroscopic imaging is performed from two orthogonal directions, and the object's position is finely adjusted while the position of the teeth is visually checked on the fluoroscopic image.
(4) CT image data is acquired by rotating the first rotation system 6 (rotative arm 2) while the cone beam X-ray 3b is irradiated.
(5) The acquired image data is subjected to calculation processing in image processing device 12 to reconstruct a two-dimensional tomographic image or a three-dimensional image, and the image is displayed on image display device 13.

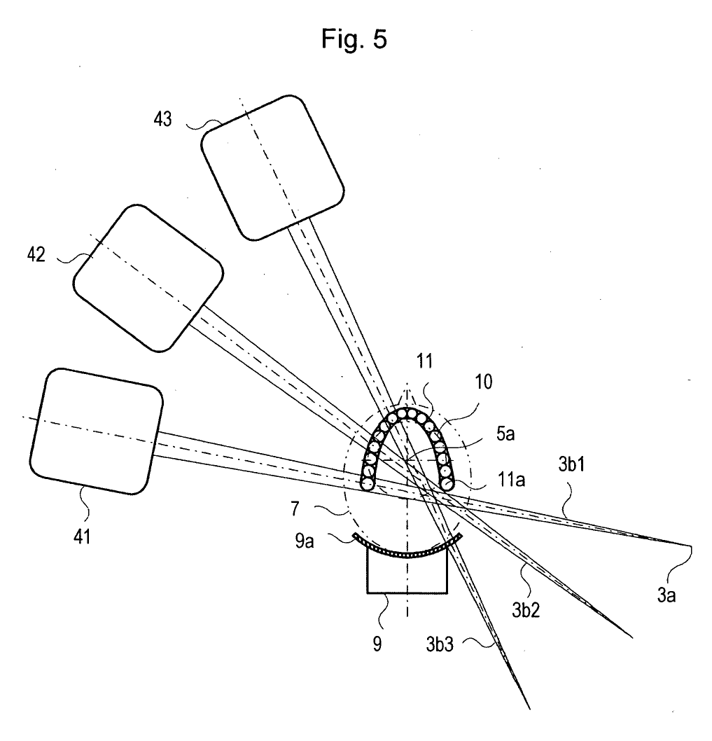

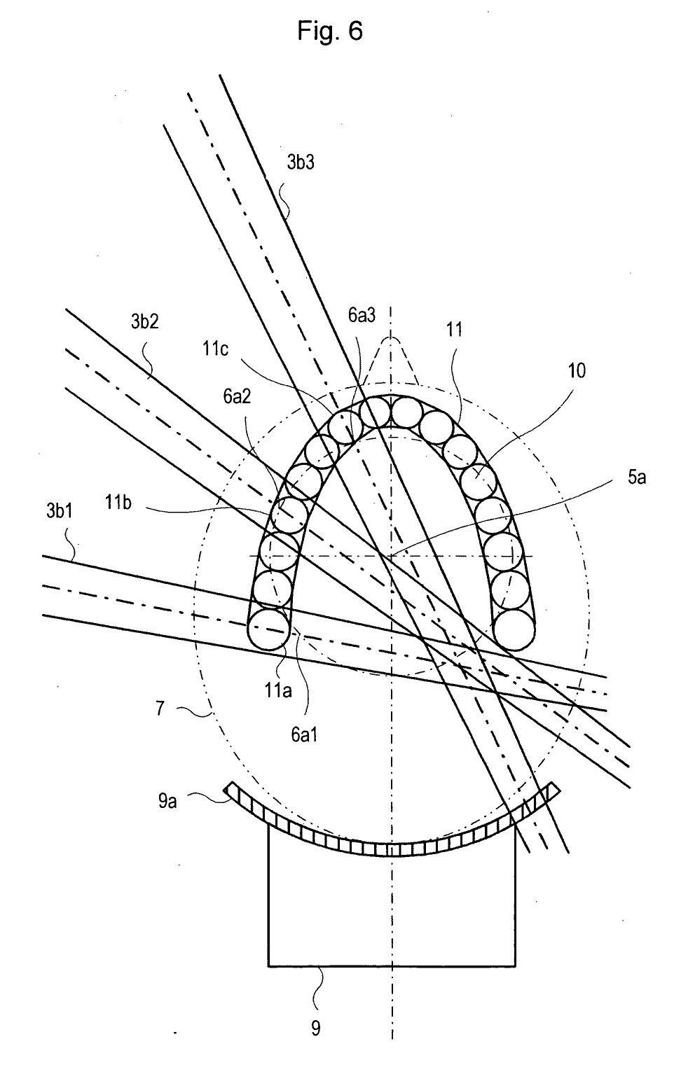

FIG. 5 is a diagram showing the operation when panoramic imaging is performed by the X-ray CT apparatus according to this embodiment, and FIG. 6 is a partial enlarged view thereof. First, the center in the left-right direction of the object 7 is paced in agreement with the rotation center 5a of the second rotation system 5. If the center in the left-right direction of chair 8 is preset to be just below the rotation center 5a of second rotation system 5, position adjustment to the object 7 in the left-right direction is scarcely necessary, except for fine adjustment of the head holder 9. However, it is here postulated that the dental arc 11 of the object 7 is symmetrical with respect to the center of the object 7 in the left-right direction. Subsequently, the position of the object 7 in the back-front direction is properly adjusted by combining the angle adjustment to chair back 8b of chair 8 and the position adjustment to up-down movement mechanism 8a in the up-down direction, so as to substantially match the shape and size of dental arc 11 with circle 10, the rotation radius of which corresponds to the distance d between rotation center 5a of the second rotation system 5 and the rotation center 6a of the first rotation system 6. After fixing the rear of the head to the head holder 9, the position thereof is finely adjusted by a back-front and left-right movement mechanism of head holder 9. In this manner, the rotation center 6a of the first rotation system 6 is located on circle 10.

As described above, in the state in which the position of the object 7 is fixed, rotative arm 2 is revolved by first rotation system 6 in accordance with the rotation angle of the second rotation system 5 while the second rotation system 5 is revolved. A tomogram of the dental arc 11 is obtained by irradiating the cone beam X-ray 3b in a direction perpendicular to each tooth of the dental arc 11 which does not interfere with an opposite tooth of the dental arc 11. Because of the differences in the shape and size between the actual dental arc 11 and the circle 10, which has a rotation radius corresponding to the distance d between rotation center 5a of the second rotation system 5 and the rotation center 6a of first rotation system 6, the distance between X-ray generation source 3a and the object 7 varies depending on the position of the tooth being examined. As a result, the expansion ratio and the density of the fluoroscopic image projected to the two-dimensional X-ray detector 4 will vary. That is, when a tooth 1 la is imaged, although the cone beam X-ray 3b1 irradiated from the X-ray generator 3 is transmitted through the center of rotation center 6a1 of the first rotation system 6 and the tooth 11a, there is a little distance between the rotation center 6a1 of the first rotation system 6 and the center of the tooth 11b.

In a similar manner, when tooth 11b is imaged, although cone beam X-ray 3b2, which is irradiated from the X-ray generator 3 is transmitted through rotation center 6a2 of first rotation system 6 and the center of tooth 11b, there is a little distance between rotation center 6a2 of first rotation system 6 and the center of tooth 11b. When tooth 11c is imaged, although cone beam X-ray 3b3, which is irradiated the X-ray generator 3, is transmitted through rotation center 6a3 of first rotation system 6 and the center of tooth 11c, there is a little distance between rotation center 6a3 of first rotation system 6 and the center of tooth 11c.

Accordingly, after the differences therebetween are corrected in the image calculating processing in synchronism with the rotation angle of second rotation system 5, partial image data at each angle are joined to reconstruct a continuous image over the whole angle. Thus, an accurate panoramic image can be obtained.

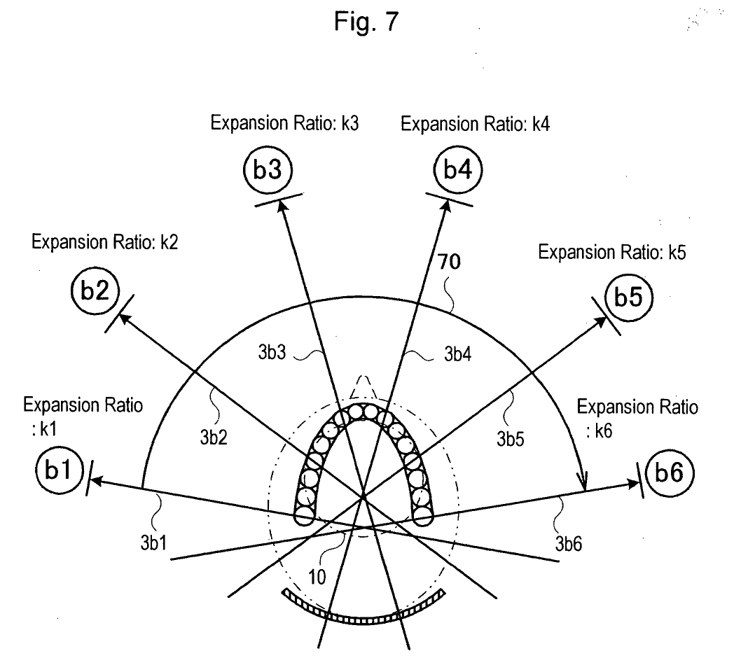

FIG. 7 is a diagram showing the differences between the rotation center of first rotation system 6 and the center of the respective teeth in the case where the rotation angle of the second rotation system 5 is varied each time. As the imaging of a tooth is gradually shifted from the left end to the right end of the dental arc, the rotation center 6a of first rotation system 6 moves on circle 10. Accordingly, the irradiation angle of the cone beam X-rays 3b1 to 3b6 is gradually as depicted by varied arrow 70. By irradiating cone beam X-rays 3b1 to 3b6, fluoroscopic images b1 to b6 are obtained by the two-dimensional X-ray detecting device 42. Because of the differences between the shape and size of the actual dental arc and those of circle 10, which has a rotation radius corresponding to the distance d between rotation center 5a of the second rotation system 5 and the rotation center 6a of the first rotation system 6, a difference occurs in the distance between X-ray generator 3a and the object 7 depending on the position of the tooth being examined. Resultingly, a difference occurs in the expansion ratio and density of the fluoroscopic image projected on two-dimensional X-ray detecting device 4.

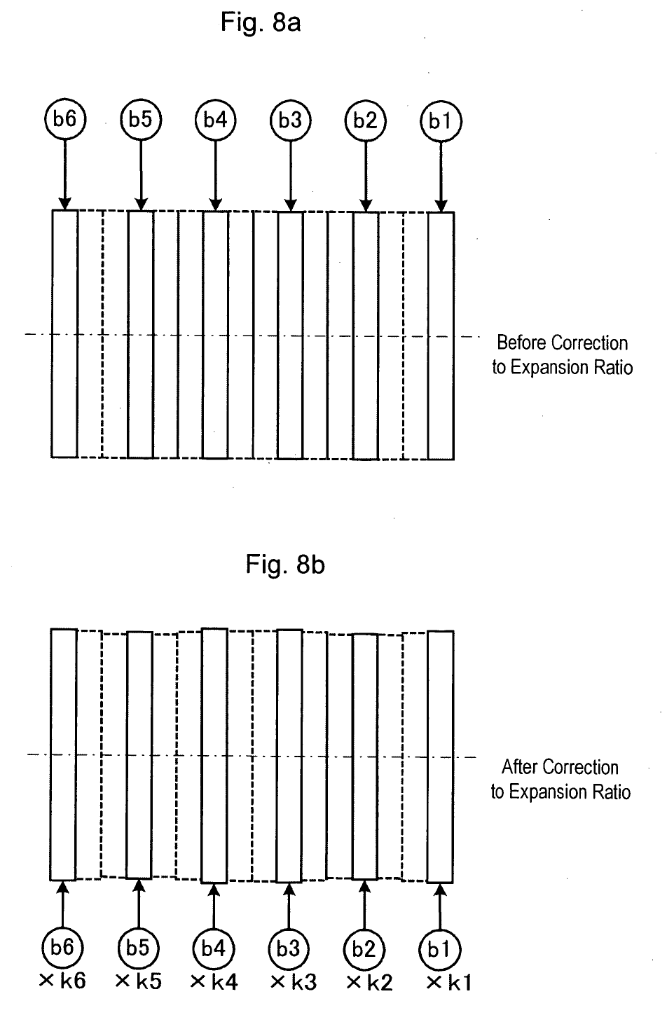

FIGS. 8a and 8b are diagrams showing the state of fluoroscopic images b1 to b6 obtained by cone beam X-rays 3b1 to 3b6 before and after correction of expansion ratio, respectively. As shown in FIG. 8a, each of the fluoroscopic images b1 to b5 before the correction of the expansion ratio substantially has the same size. By multiplying those fluoroscopic images b1 to b6 before correction by expansion ratios k1 to k6, respectively, in accordance with the difference between the rotation center of first rotation center 6 (point on circle 10) and the center of each tooth, the sizes of the fluoroscopic images b1 to b6 can be corrected, as shown in FIG. 8b. Then, a panoramic image is reconstructed on the basis of the size-corrected image. Meanwhile, although the density of the image is not shown in the figure, it is needless to say that the density is also corrected.

In this correction processing, the amount of information to be dealt with is small, and so the correction processing itself is simple.

Further, if two kinds of dental arcs of standard size are prepared respectively for adults and children as the shape and size of dental arc 11 to serve as a reference in the correction, the correction can be automatically done with reference thereto and only two kinds of tables of correction coefficient used with the software are necessary. Accordingly, it is possible to reduce the memory capacity installed in the image processing device 12 and shorten the time taken for image processing. If the shape and size of the dental arc 11 customized for each individual can be produced other than those of the reference size for the software, it is needless to say that more accurate correction can be done.

The execution of the above panoramic imaging procedure is outlined in the following order of steps (1) to (6):
(1) By combining the angle adjustment of the chair back 8b of chair 8 and the up-down position adjustment of the up-down movement mechanism 8a, the imaging region of object 7 is positioned so that the trajectory 10 (circular trajectory) drawn by rotation center 6a of rotative arm 2 (first rotation system 6) substantially coincides with the dental arc 11.
(2) The head holder 9 of chair 8 is fine adjusted to fix the position of object 7.
(3) The second rotation system 5 is revolved to adjust one end of dental arc 11 (back tooth) to an irradiation starting position. At the same time, the rotation angle of the rotative arm 2 is adjusted in a direction perpendicular to dental arc 11 which does not interfere with an opposite tooth of the dental arc 11, and irradiation of cone beam X-ray 3b is started.
(4) While the second rotation system 5 is revolved along dental arc 11, the rotation angle of the rotative arm 2 is adjusted in a direction perpendicular to the dental arc 11, which does not interfere with each of the opposite teeth of dental arc 11, cone beam X-ray 3b is sequentially irradiated, and thus partial fluoroscopic image data is acquired at each rotation angle of second rotation system 5.
(5) The imaging procedure is completed when the position of the data acquisition of a partial fluoroscopic image reaches the other end of the dental arc (back tooth opposite to the starting position).
(6) After the expansion ratio and density of the partial fluoroscopic image data acquired at each rotation angle of the second rotation system 5 are corrected in the image processing device 12 in synchronism with each rotation angle of the second rotation system 5, a continuous panoramic image of the dental arc 11 over the whole angle is reconstructed and presented on image display device 13.

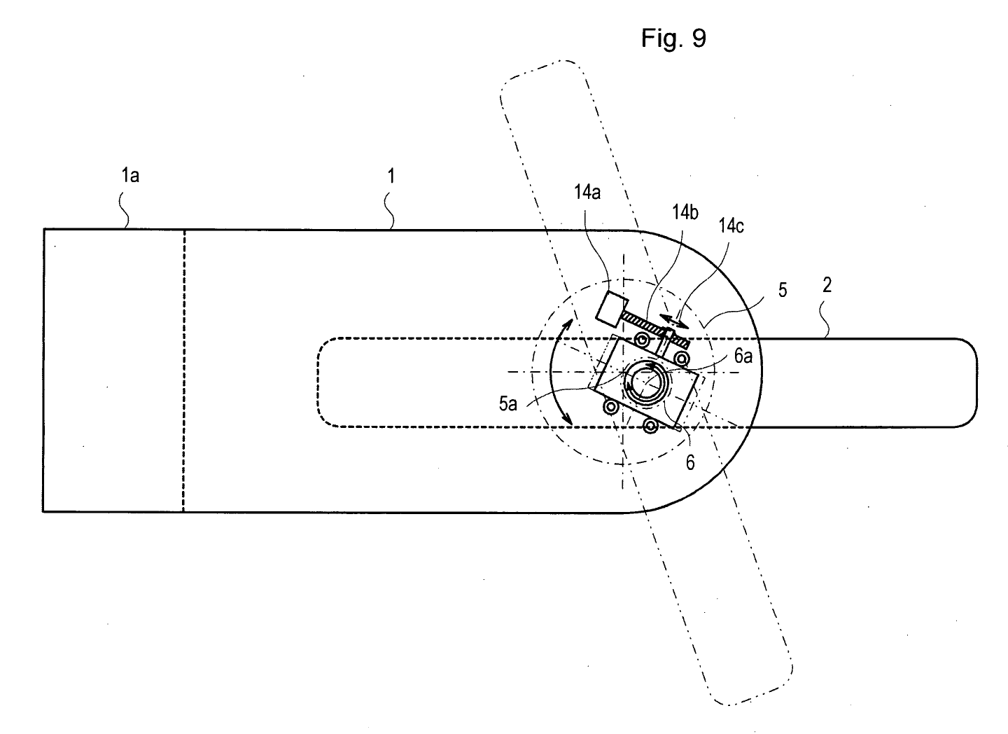

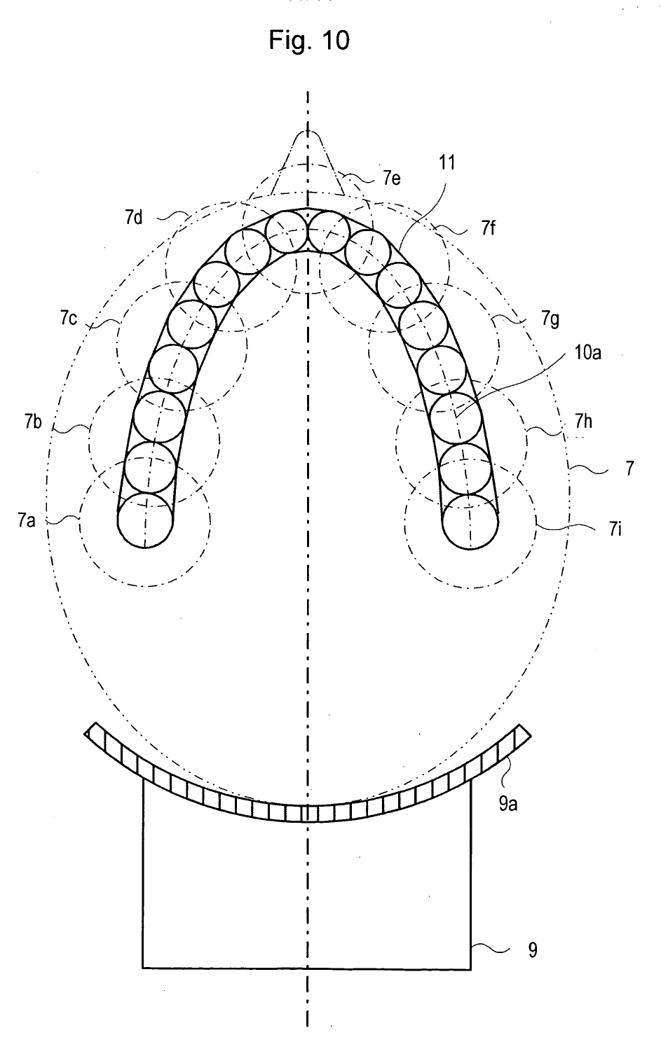

FIG. 9 is a diagram showing a variation of the rotation mechanism of the second rotation system of the X-ray CT apparatus according to the present invention, and it is provided as a top view of the X-ray CT apparatus of FIG. 1. FIG. 10 is a diagram showing a rotation radius drawn by this X-ray CT apparatus, and it represents an enlarged view corresponding to FIG. 4. Although the distance (rotation radius) d between the rotation center 5a of the second rotation system 5 and the rotation center 6a of the first rotation system 6 is fixed in the X-ray CT apparatus shown in FIG. 1, in the X-ray CT apparatus shown in FIG. 9, the rotation radius d can be freely changed, and the rotation center 6a of first rotation system 6 moves on a complicated trajectory along dental arc 11. A linear driving system includes driving means 14a, such as a servomotor, mounted on the second rotation system 5, and a linear driving mechanism 14b. such as a feed screw and a rack and pinion, driven by driving means 14a. In the linear driving system, the rotation center 6a of the first rotation system 6 is moved in the direction of arrow 14c, and thus the distance (rotation radius) d between the rotation center 5a of the second rotation system 5 and the rotation system 6a of the first rotation system 6 is moved to a desired position. In this manner, by use of the linear driving system for correcting the position of the rotation center 6a of the first rotation system 6, the trajectory of the rotation center 6a of the first rotation system 6 is drawn on a curved line 10a taken along dental arc 11, as shown in FIG. 10. Accordingly, because a difference between the rotation center 6a of first rotation system 6 and the center of teeth 11 does not occur, correction processing in the image calculating processing becomes unnecessary, and so the calculation time can be shortened.

The moving range of the rotation center 6a of the first rotation system 6, which is moved by the linear driving system, is a difference corresponding to the distance between the center of each tooth of the dental arc 11 and the rotation radius 10 in FIG. 4, around ±15 mm being enough. Further, if the moving distance is smaller, a load supporting/driving device can be miniaturized, and bending and the like occurring due to the weight of apparatus can be reduced. Therefore, position adjustment can be accurately carried out using a simple mechanism.

According to the X-ray CT apparatus shown in FIG. 9, accurate positioning is easily performed in the apparatus, regardless of the shape and size of the dental arc of the object. If the apparatus is constructed so that this adjusting mechanism of the rotation radius can be remote controlled, fine adjustment of the head holder 9 becomes unnecessary, whereby it is possible to greatly reduce the burden on the object 7 and to simplify the mechanism for adjusting the position of chair 8 and the fixing object 7. Further, image correction in regard to differences in the expansion ratio and the density of a fluoroscopic image obtained in the panoramic imaging also becomes unnecessary, whereby the time taken for the image calculation processing can be shortened.

Since it is not necessary to move the object with use of this X-ray CT apparatus, the following imaging method can be conducted. That is, as shown in FIG. 10, X-ray CT imaging is sequentially and repeatedly executed plural times (nine times in FIG. 10) on local regions 7a to 7i, each including two to three teeth, so as to cover the whole dental arc 11, and thus CT image data combining a plurality of local imaging regions 7a are acquired. In this manner, even when an X-ray detecting device having a small FOV is used, CT image data for presenting the whole dental arc can be acquired.

The execution of the above imaging procedure on local region can be outlined in the following order of steps (1) to (7):
(1) An imaging region of object 7 is positioned and fixed so that a trajectory drawn by rotation center 6a of the rotative arm 2 (dental arc-shaped trajectory) coincides with the dental arc 11 of object 7.
(2) The rotation center 6a of the rotative arm 2 is adjusted to the center of a back tooth located at one end of dental arc 11, i.e. local region 7a.
(3) A cone beam X-ray 3b is irradiated while the rotative arm 2 is revolved, and thus CT image data is acquired.
(4) The second rotation system 5 is revolved to adjust the rotation center 6a of the rotative arm 2 to the center of local region 7b adjacent to and partially overlapping with local region 7a, the CT image data of which was acquired above.
(5) CT image acquisition and positioning are repeatedly carried out on local regions 7b to 7i along dental arc 11.
(6) The imaging procedure is completed when CT image data acquisition on the other end of the dental arc 11 (center of back tooth on the end opposite to the starting position, i.e. local region 7i) is finished.
(7) The CT image data acquired is subjected to calculation processing in the image processing device 12, and an image of the whole dental arc 11 is reconstructed and displayed on the image display device 13.

According to this method of imaging local regions, an image of higher resolution can be obtained on an identical region. Further, it is also possible to extract fluoroscopic image data along the dental arc in a direction perpendicular to the tooth row from the above-mentioned data and to reconstruct a panoramic image. Similarly, a tomographic image and a three-dimensional image of an arbitrary cross section can be reconstructed. Further, by limiting the X-ray irradiating region to a local region, the exposure dose of object 7 can be reduced. According to this embodiment, the exposure dose can be reduced as the number of times of CT imaging becomes smaller.

When the method of imaging a local region is applied to the imaging method of FIG. 3 to FIG. 6, it is needless to say that similar imaging can be performed by sequentially repeating the local CT imaging, while positioning the patient at every CT imaging on the local region.

Meanwhile, although the X-ray CT apparatus according to this embodiment is suitable for dentistry, it is needless to say that this technique is not limited to dentistry and is applicable to a general X-ray CT apparatus. For example, the method is applicable when an object to be examined is larger than the imaging range of the X-ray CT apparatus, as well as in the case where sequential local CT imaging is performed on the whole object, or in the case where panoramic imaging is performed from the inside of a cylindrical body simulating the shape of a dental arc.

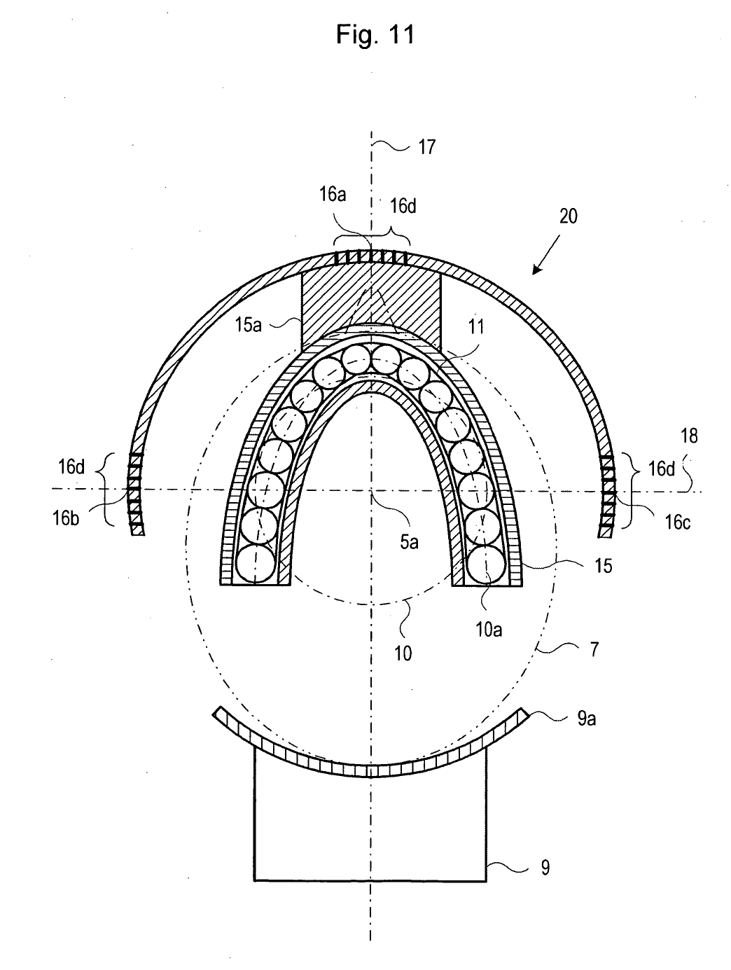

FIG. 11 is a diagram showing the structure of a positioning device used in the X-ray CT apparatus according to this embodiment. The positioning device 20 includes a dental articulation unit 15 produced in accordance with a dental model of each object to be examined and a flange 16 joined to the dental articulation unit 15 via a joint unit 15a. Flange 16 is made of a thin plate fixed onto a surface parallel to the dental articulation unit 15. When the dental articulation unit 15 is placed in the object's mouth, flange 16 is exposed outside the mouth of the object 7 via joint unit 15a. On flange 16, line marks 16a to 16c are incused along orthogonal axes 17 and 18. Further, on both sides of the respective line marks 16a to 16c, scale marks 16d are incused at regular intervals around line mark 16a to 16c. Scale mark 16d gives an indication of the distance when the object's position is shifted in CT imaging so as to match the center of the region imaged with rotation center 6a of the rotative arm 2.

The X-ray CT apparatus is provided with a projector (not shown) for projecting an optical marker to the face of object 7 from three directions corresponding to the line marks 16a to 16c of the flange 16, which is positioned so that an intersecting point of those light axes passes through rotation center 5a of the second rotation system 5. Then, by fine adjusting the position of the object 7 wearing the positioning device 20 so that line marks 16a to 16c coincide with the optical marker, the position of the rotation center 5a of the second rotation system 5 can be visually checked from the outside of the mouth. Accordingly, accurate positioning can be realized only with positioning based on the optical marker, without performing X-ray fluoroscopic imaging.

By changing the position of attachment of the joint unit 15a and flange 16, the positional relation between the dental articulation unit 15 and the rotation center 5a of the second rotation system 5 can be changed. That is, an adjustment can be performed so that the trajectory (circular trajectory) 10 drawn by rotation center 6a of the rotative arm 2 substantially coincides with the dental arc 11. In this adjustment, it is useful to prepare in advance a full-scale figure like the one shown in FIG. 11 for checking the state of correspondence of the circular trajectory 10 and the dental arc 11 and to combine them while checking that the figure corresponds with the real thing. Further, this figure also may be used when it is checked that the intersecting point of the light axes of the optical marker projector from three directions passes through the rotation center 5a of the second rotation system 5. Meanwhile, although the above description relates to a case in which positioning device 20 is applied to the X-ray CT apparatus shown in FIG. 3 to FIG. 6, it also may be applied similarly to the X-ray CT apparatus shown in FIGS. 9 and 10 by using a dental arc-shaped trajectory 10a, instead of the circular trajectory 10. Further, the above-described positioning processing may be automatically carried out by detecting positioning device 20 using a camera or the like and performing image processing.

The execution of the imaging procedure using the above-described positioning device 20 is outlined in the following order of steps (1) to (4):
(1) Positioning device 20 is worn by object 7, in which dental articulation unit 15 is produced on the basis of a dental model of the object 7.
(2) The position of attachment of the joint unit 15a and flange 16 is adjusted to connect and fix them so that the trajectory (circular trajectory) 10 drawn by the rotation center 6a of the rotative arm 2 or the dental arc-shaped trajectory 10a substantially coincides with the dental arc 11.
(3) Dental articulation unit 15 of the device joined and fixed is put on to dental arc 11.
(4) Line marks 16a to 16c of the flange 16 are made corresponding with the optical marker of the apparatus and the object is positioned.

According to the above-described embodiment, in the cone beam X-ray CT imaging apparatus for dentistry, positioning in local CT imaging can be easily carried out with a simple rotation mechanism, and the positioning mechanism can be simplified. Further, panoramic imaging also can be performed easily, and it is possible to greatly shorten the time for image calculation processing and to simplify the image processing device. Further, it is possible to perform local CT imaging and panoramic imaging without moving the object and to simplify the position adjustment of the chair and the object fixing mechanism. Further, image correction in regard to differences in the expansion ratio and density of a fluoroscopic image of an imaging region becomes unnecessary. In an X-ray CT apparatus with a small FOV, too, CT image data of high resolution over the whole dental arc can be acquired by sequentially repeating the local CT imaging plural times. Further, a panoramic image of the whole dental arc, a tomographic image of an arbitrary cross section, and a three-dimensional image can be reconstructed from those image data.

Meanwhile, according to the above-described embodiment, rotative arm 2 is constructed as a single unit. However, it is also available to construct the rotative arm as a dual unit and relatively slide each unit so as to freely extend and contract its length in its radius direction and adjust the distance between the X-ray generating device 3 and the two-dimensional X-ray detecting device 4.

### INDUSTRIAL APPLICABILITY

As described above, in an X-ray CT apparatus according to the present invention, when a cone beam X-ray is irradiated to the whole jaw, including a dental arc, and to a local region, such as a tooth and around the jaw joint, and a panoramic image showing conditions of the dental row, teethridge, and the tissue and bone around them is obtained, the time taken for image processings can be extremely shortened.

1. An X-ray CT apparatus comprising:
X-ray generating means for generating an X-ray;
X-ray detecting means arranged opposite to the X-ray generating means for two-dimensionally detecting an X-ray dose which is transmitted through an object to be examined;
holding means for holding the X-ray generating means and the X-ray detecting means so that the object is positioned therebetween;
first rotation driving means for driving the holding means to rotate on a locus of movement around the object;
support means for supporting the first rotation driving means;
image processing means for producing an image of the object on the basis of the X-ray dose detected by the X-ray detecting means; and
image display means for displaying the image produced in the image processing means,
further comprising;
second rotation driving means to integrally rotate the holding means supported by the support means and the support means in a manner such that the rotation center of the second rotation driving means is parallel with the rotation center of the first rotation driving means and is located at a different position from that of the first rotation driving means; and
drive control means for controlling the first rotation driving means in a first imaging mode and separately controlling the first rotation driving means and the second rotation driving means in a second imaging mode.

2. An X-ray CT apparatus according to claim 1, wherein the drive control means performs control for execution of each of the first imaging mode and the second imaging mode.

3. An X-ray CT apparatus according to claim 1, wherein the image processing means reconstructs a two-dimensional tomographic image or a three-dimensional image of the object in the first imaging mode, and reconstructs a panoramic image of the object in the second imaging mode.

4. An X-ray CT apparatus according to claim 1, wherein each of the rotation center of the first rotation driving means and the second rotation driving means is arranged so that the distance therebetween is determined on the basis of the size of an imaging region of the object.

5. An X-ray CT apparatus according to claim 1, wherein the rotation angle of the second rotation driving means is determined so that each of the holding means and the first rotation driving means is located in a predetermined imaging region of the object.

6. An X-ray CT apparatus according to claim 1, wherein, in the case of executing the second imaging mode, differences in expansion ratio of a fluoroscopic image of the object, which occur due to differences in the distance between the locus of movement of the center of the first rotation driving means and each imaging region of the object, are corrected by an image calculating processing.

7. An X-ray CT apparatus according to claim 1, wherein said holding means in a rotative arm; and the second rotation driving means is rotatable around a center of rotation of the rotative arm on a locus of movement simulating the shape of an imaging region of the object, positions the local X-ray irradiating region in the first imaging mode, and adjusts the imaging direction in the combination of the position of the irradiating region and a rotation angle of the rotative arm in the second imaging mode.

8. An X-ray CT apparatus according to claim 1, wherein said holding means is a rotative arm; and the second rotation driving means is rotatable around a center of rotation of the rotative arm on a circumference simulating the shape of an imaging region of the object, and has a mechanism for varying the diameter of the locus of movement of the center of the first rotation driving means.

9. An X-ray CT apparatus according to claim 1, wherein by locally repeating the first imaging mode plural times along the imaging region of the object with the second rotation driving means, image data in the first imaging mode over the imaging region of the object are acquired, and a panoramic image over the imaging region, a tomographic image or a three-dimensional image of an arbitrary cross section of the object is reconstructed from the image data.
